(19) United States
(12) Patent Application Publication
(10) Pub. No.: US 2023/0045066 A1
(43) Pub. Date: Feb. 9, 2023

(54) BIOMARKERS FOR DIAGNOSING BREAST CANCER AND USES THEREOF

(71) Applicant: SEOUL NATIONAL UNIVERSITY R&DB FOUNDATION, Seoul (KR)

(72) Inventors: Je Yoel CHO, Seoul (KR); Hyoung Min PARK, Daejeon (KR)

(21) Appl. No.: 17/393,836

(22) Filed: Aug. 4, 2021

(51) Int. Cl. G01N 33/574; C12Q 1/6886

(52) U.S. Cl. CPC G01N 33/57415; C12Q 1/6886; G01N 2333/755; G01N 2333/936; G01N 2333/7458; G01N 2333/78; G01N 2333/4713; G01N 2333/4716; C12Q 2600/158

(57) ABSTRACT

Provided are a composition for diagnosing breast cancer and a method of diagnosing breast cancer using the same. Breast cancer may be diagnosed using blood in a simple manner.

### BACKGROUND

### 1. Field

The present disclosure relates to biomarkers for diagnosing breast cancer and uses thereof.

### 2. Description of the Related Art

There are many similarities between dogs and humans in mammary gland cancer and breast cancer. Dogs are chosen as animal models for human diseases, such as cancer, because they are exposed to human environments and spontaneously develop diseases more rapidly than humans. In particular, it is known that environmental stimuli may have profound effects on epigenetics of an organism, and it has been reported that epigenetic abnormalities of several genes are associated with the development of human breast cancer.

Therefore, since investigating the epigenetics of canine mammary gland cancer may be very useful in terms of comparative oncology, it is necessary to develop a diagnostic method commonly applied to dogs and humans by using diagnostic markers developed with a comparative medical approach.

### SUMMARY

An aspect provides a composition for diagnosing breast cancer, the composition including an agent for measuring an expression level of LPC1 protein or mRNA of a gene thereof.

Another aspect provides a kit for diagnosing breast cancer, the kit including the composition.

Still another aspect provides a method of providing information for diagnosing breast cancer, the method including measuring an expression level of LPC1 protein or mRNA of a gene thereof in a biological sample of an individual.

Additional aspects will be set forth in part in the description which follows and, in part, will be apparent from the description, or may be learned by practice of the presented embodiments of the disclosure.

An aspect provides a composition for diagnosing breast cancer, the composition including an agent for measuring an expression level of LPC1 protein or mRNA of a gene thereof. In a specific embodiment, the composition may further include an agent for measuring an expression level of SERPING1, C1QA, LYZ, SEPP1, FN1, PROS1, VWF, SHBG, CFD, PRDX2, or HABP2 protein, or mRNA of a gene thereof.

In one embodiment, biomarkers associated with breast cancer were discovered, based on proteomics, and it was confirmed that human breast cancer development may be predicted and diagnosed, based on changes in protein expression in dogs with mammary gland cancer. In other words, biomarkers associated with breast cancer of dogs and their guardians who share the same living environment were identified through epigenetics, and a diagnostic method commonly applicable to dogs and humans was confirmed. Therefore, the composition for diagnosing breast cancer according to an aspect may be used to predict or diagnose breast cancer of a guardian from a dog having mammary gland cancer. In one embodiment, common proteins with increased expression in companion dogs with mammary gland cancer and patients with breast cancer, as compared with a normal control group, were also identified. Therefore, the proteins may be used as biomarkers for diagnosing canine mammary gland cancer and human breast cancer.

As used herein, the term “breast cancer” refers to cancer occurring in the breast, and may be also interchangeably referred to as “mammary gland cancer”. The breast cancer may include mammary gland breast cancer, lobular breast cancer, or a combination thereof.

As used herein, the term “diagnosis” refers to identification of the presence or characteristics of pathophysiology. In one aspect, the diagnosis is to determine occurrence and/or severity of breast cancer, i.e., a progression status, and includes a prognosis after treatment or surgery, i.e., a possibility of recurrence.

As used herein, the term “marker” refers to a substance that may distinguish and diagnose an individual having breast cancer from a normal individual, and the marker includes all organic biomolecules such as polypeptides, proteins or nucleic acids, genes, lipids, glycolipids, glycoproteins, sugars, etc., which show an increase or a decrease in an individual having breast cancer of the present disclosure. Particularly, in the present disclosure, the marker may be a protein or gene changed in a tissue isolated from an individual having breast cancer.

As used herein, the term “agent for measuring an expression level of a protein or mRNA thereof” refers to a molecule that may be used for detection and/or quantitation of a marker by examining an expression level of LPC1, SERPING1, C1QA, LYZ, SEPP1, FN1, PROS1, VWF, SHBG, CFD, PRDX2, or HABP2 protein, or mRNA thereof, each protein is a marker increased or decreased in patients with breast cancer. Specifically, an agent for measuring a protein expression level of the marker may be, for example, an antibody, an antibody mimetic, an aptamer, an avimer (avidity multimer), or a peptidomimetic specifically binding to a protein encoded by the marker gene or a fragment thereof, and the antibody may be a polyclonal antibody or a monoclonal antibody.

As used herein, the term “antibody” may refer to a specific protein molecule directed against an antigenic domain. With respect to the objects of the present disclosure, the antibody refers to an antibody that specifically binds to a marker protein, and includes a polyclonal antibody, a monoclonal antibody, and a recombinant antibody. Preparation of antibodies may be easily performed using techniques widely known in the art. In addition, the antibody of the present disclosure includes a functional fragment of an antibody molecule as well as a complete form having two full-length light chains and two full-length heavy chains. A functional fragment of an antibody molecule refers to a fragment having at least an antigen-binding function, and includes Fab, F(ab′), F(ab′)2, and Fv.

An agent for measuring an mRNA expression level of the gene may be, for example, a primer pair, probe, or antisense oligonucleotide, which specifically binds to the gene. In this regard, since the nucleic acid information of the genes has been known in Genebank, etc., primers or probes, which specifically amplify a specific region of the gene, may be designed by those of ordinary skill in the art, based on the sequence.

As used herein, the term “primer pair” refers to any combination of a primer pair consisting of forward and reverse primers that recognize a sequence of a target gene, and particularly, is a primer pair that gives analysis results with specificity and sensitivity. Because a nucleotide sequence of a primer does not match a non-targeted sequence in a sample, the primer may show high specificity when it amplifies only a target gene sequence containing a complementary primer binding site without causing non-specific amplification.

As used herein, the term “probe” refers to a substance capable of specifically binding to a target substance to be detected in a sample, and refers to a substance capable of specifically identify the presence of the target substance in the sample via the binding. The kind of the probe molecule is a substance commonly used in the art, and it may be specifically, but is not particularly limited to, peptide nucleic acid (PNA), locked nucleic acid (LNA), a peptide, a polypeptide, a protein, RNA, or DNA. More specifically, the probe may be a biomaterial derived from an organism, an analogue thereof, or a material prepared ex vivo, and may include, for example, enzymes, proteins, antibodies, microorganisms, animal/plant cells and organs, neural cells, DNA, and RNA. DNA may include cDNA, genomic DNA, and oligonucleotides, RNA may include genomic RNA, mRNA, and oligonucleotides, and proteins may include antibodies, antigens, enzymes, peptides, etc.

As used herein, the term “antisense oligonucleotide” refers to DNA or RNA or a derivative thereof containing a nucleic acid sequence complementary to a specific mRNA sequence, and binds to the complementary sequence within mRNA to inhibit translation of mRNA into protein. The antisense oligonucleotide sequence may be a DNA or RNA sequence that is complementary to mRNA of the gene, and is able to bind to the mRNA. It is able to inhibit translation, cytoplasmic translocation, or maturation of mRNA of the gene or all other activities essential for overall biological functions. The antisense oligonucleotide has a length of 6 to 100 bases, specifically, 8 to 60 bases, and more specifically 10 to 40 bases. The antisense oligonucleotide may be synthesized in vitro by an ordinary method and administered to the body, or may be allowed to be synthesized in vivo. An example of synthesizing the antisense oligonucleotide in vitro is to employ RNA polymerase I. An example of synthesizing the antisense RNA in vivo involves performing transcription of antisense RNA using a vector containing a multicloning site (MCS) in the opposite direction. Such antisense RNA preferably contains a translation stop codon in its sequence to block translation into a peptide sequence.

Another aspect provides a kit for diagnosing breast cancer, the kit including the composition. A specific description of the composition is the same as described above. Specifically, the kit may diagnose breast cancer by determining a protein expression level of LPC1, SERPING1, C1QA, LYZ, SEPP1, FN1, PROS1, VWF, SHBG, CFD, PRDX2, or HABP2 which is a diagnostic marker for breast cancer by measuring an expression level of mRNA of the gene or the protein. The kit may include an agent for measuring the mRNA expression level of the diagnostic marker gene for breast cancer, e.g., a primer pair, a probe, or an antisense nucleotide specifically binding to the gene, and an agent for measuring a protein expression level, e.g., an antibody specifically binding to the marker protein or an immunobinding fragment thereof.

The kit of one specific embodiment may be a reverse transcription polymerase chain reaction (RT-PCR) kit, a DNA chip kit, an enzyme-linked immunosorbent assay (ELISA) kit, a protein chip kit, a rapid kit, or a multiple reaction monitoring (MRM) kit.

Further, the kit for diagnosing breast cancer may be configured by further including a composition, a solution, or an apparatus consisting of one or more kinds of components suitable for an analysis method. For example, the diagnostic kit may be a diagnostic kit including necessary components for performing reverse transcription polymerase chain reaction. The reverse transcription polymerase chain reaction kit includes a primer pair specific to each marker gene. The primer is a nucleotide having a sequence specific to a nucleic acid sequence of each gene, and has a length of about 7 bp to about 50 bp, and more specifically, a length of about 10 bp to about 30 bp. Further, a primer specific to a nucleic acid sequence of a control gene may be included. The reverse transcription polymerase chain reaction kit may include a test tube or different suitable container, reaction buffers (varying in pH and magnesium concentrations), deoxyribonucleotides (dNTPs), enzymes such as Taq-polymerase and reverse transcriptase, a DNase, an RNase inhibitor, DEPC-water, sterile water, etc. Further, the DNA chip kit may include, for example, a substrate onto which cDNAs or oligonucleotides corresponding to the genes or fragments thereof are attached, and reagents, agents, and enzymes for preparing fluorescent probes, etc. Further, the substrate may include cDNAs or oligonucleotides corresponding to control genes or fragments thereof. Further, the kit may be, for example, a diagnostic kit including essential elements needed to perform ELISA. The ELISA kit include antibodies specific to the proteins. The antibodies may be monoclonal, polyclonal or recombinant antibodies, which have high specificity and affinity to each marker protein and have little cross-reactivity to other proteins. In addition, the ELISA kit may include an antibody specific to a control protein. The ELISA kit may further include reagents capable of detecting bound antibodies, e.g., a labeled secondary antibody, chromophores, enzymes (e.g., conjugated with an antibody) and their substrates or other substances capable of binding to the antibodies. Further, the kit may be, for example, a rapid kit including essential elements needed to perform a rapid test to know analysis results. The rapid kit includes an antibody specific to the protein. The antibodies may be monoclonal, polyclonal or recombinant antibodies, which have high specificity and affinity to each marker protein and have little cross-reactivity to other proteins. In addition, the rapid kit may include an antibody specific to a control protein. In addition, the rapid kit may include other materials such as reagents capable of detecting bound antibodies, e.g., a nitrocellulose membrane onto which a specific antibody and a secondary antibody are immobilized, a membrane to which antibody-conjugated beads are bound, an absorption pad, a sample pad, etc. Further, the kit may be, for example, a multiple reaction monitoring (MRM) kit in an MS/MS mode including essential elements needed to perform mass spectrometry. MRM is a method of using ions obtained by selecting specific ions from broken ions and bombarding the selected ions through the source of another consecutively connected MS, whereas selected ion monitoring (SIM) is a method of using ions produced by bombardment on the source region of a mass spectrometer. Through the MRM analysis method, a protein expression level of a normal control group or the same individual may be compared with a protein expression of an individual with breast cancer, and occurrence of breast cancer may be diagnosed by examining whether the expression levels of proteins from the breast cancer marker genes are significantly increased or decreased.

Still another aspect provides a method of providing information for diagnosing breast cancer, the method including measuring an expression level of LPC1, SERPING1, C1QA. LYZ. SEPP1. FN1, PROS1, VWF, SHBG, CFD, PRDX2, or HABP2 protein or mRNA thereof in a biological sample isolated from an individual; and comparing the measured expression level with an expression level thereof in a normal control group.

In one specific embodiment, the method includes measuring an expression level of LPC1, SERPING1, C1QA, LYZ, SEPP1, FN1, PROS1, VWF, SHBG, CFD, PRDX2, or HABP2 protein or mRNA thereof in a biological sample isolated from an individual. Specifically, the expression level of the protein or mRNA thereof may be determined by measuring the mRNA expression level of the marker gene or the expression level of the protein encoded by the gene. Isolation of the mRNA or protein from the sample of the individual may be appropriately performed by those skilled in the art according to a method known in the art. For example, an individual's breast cancer tissue may be homogenized with a buffer for protein extraction or a buffer for nucleic acid extraction, followed by centrifugation, and then the obtained supernatant may be used as the sample of the individual.

The individual is a subject for detecting breast cancer, for example, a subject for predicting the likelihood of breast cancer, a subject for diagnosing the status of breast cancer, a subject for determining prognosis of breast cancer, a subject for determining a dosage of a drug for preventing or treating breast cancer, or a subject for determining a therapeutic method according to progression of breast cancer, etc. In addition, the individual refers to a guardian who lives with a companion dog and an individual who shares an environment related to daily life. The individual may be a vertebrate, specifically, a mammal, an amphibian, a reptile, a bird, etc., and more specifically, it may be a mammal, for example, a human (Homo sapiens), and a Korean. The sample may include samples such as tissues, cells, whole blood, serum, plasma, saliva, sputum, cerebrospinal fluid, or urine isolated from the individual.

The measuring of the mRNA expression level may employ, for example, reverse transcription polymerase chain reaction (RT-PCR), competitive RT-PCR, real time quantitative RT-PCR, quantitative RT-PCR, an RNase protection method, Northern blotting, or a DNA chip technology. Further, the measuring of the protein expression level may employ, for example, Western blotting, immunohistochemical staining, immunoprecipitation assay, complement fixation assay, or immunofluorescence.

In one specific embodiment, the method includes comparing the measured expression level with an expression level of a normal control group. Specifically, an expression level of the mRNA or the protein of the marker gene in a sample of a patient with breast cancer is measured, and an expression level of the mRNA or the protein of the same marker gene in a normal control group is measured, and then compared with each other, thereby determining occurrence or severity of breast cancer according to whether the expression level of the mRNA or the protein of the marker gene in the sample of the patient is higher or lower than that of the control. When the breast cancer patient is a patient after treatment or surgery, recurrence may be predicted. The above step may be, for example, determining that breast cancer is present, when an expression level of SERPING1, Cl QA. LYZ. SEPP1. FN1, PROS1, or VWF protein, or an expression level of mRNA of the gene thereof is higher than that of a normal control group. Further, the above step may be determining that breast cancer is present, when an expression level of SHBG, CFD, PRDX2 or HABP2 protein or an expression level of mRNA thereof is lower than that of a normal control group.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates a method of identifying protein information related to mammary gland cancer (breast cancer);

FIG. 2A shows a Venn diagram showing a relationship between proteins expressed in companion dogs with mammary gland cancer (cancer), normal dogs (normal), and dogs with benign tumors (benign);

FIGS. 2B and 2C show graphs showing proteins differentially expressed in companion dogs with mammary gland cancer (cancer), normal dogs (normal), and dogs with benign tumors (benign);

FIG. 3A shows a Venn diagram showing a relationship between proteins expressed in patients with breast cancer (cancer) and normal control groups (normal);

FIGS. 3B and 3C show graphs showing proteins differentially expressed in patients with breast cancer (cancer) and normal control groups (normal);

FIG. 4A shows graphs of proteins differentially expressed in companion dogs with mammary gland cancer, normal companion dogs, guardians with breast cancer, and normal control groups, respectively, as identified by a multiple mass monitoring (MRM) method;

FIG. 4B shows a Venn diagram establishing proteins common among companion dogs with mammary gland cancer, normal companion dogs, breast cancer patients, and normal control groups, respectively, after identifying proteins differentially expressed therein by volcano plots;

FIGS. 5A and 5B show expression patterns of eight kinds of proteins, of which expression was commonly increased in samples of guardians of companion dogs with mammary gland cancer, companion dogs with mammary gland cancer, and breast cancer patients; and

FIG. 5C shows a graph showing an expression pattern of LPC1, among the eight kinds of proteins, which was significantly increased in guardians of companion dogs with mammary gland cancer, as compared with guardians of normal companion dogs.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, preferred exemplary embodiments will be provided for better understanding of the present disclosure. However, the following exemplary embodiments are provided only for understanding the present disclosure more easily, but the content of the present disclosure is not limited thereby.

### EXAMPLE

### Example 1. Identification and Analysis of Canine Mammary Gland Cancer-Related Protein Information, Based on Proteomics

### 1-1. Identification of Mammary Gland Cancer-Related Proteins

Through proteomic analysis, blood proteins related to canine breast cancer (mammary gland cancer) were identified.

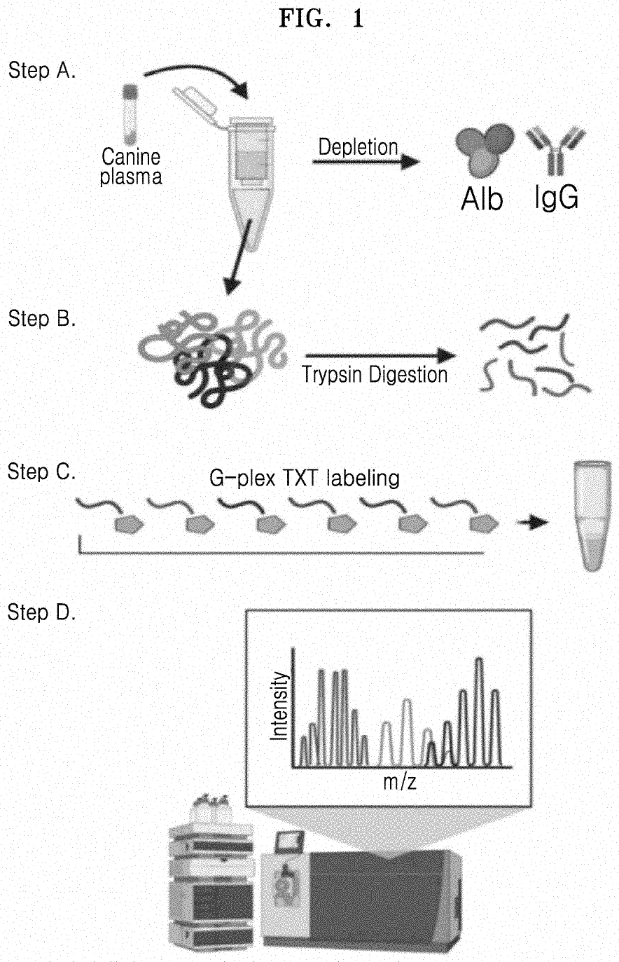

FIG. 1 illustrates a method of identifying protein information related to mammary gland cancer. In detail, companion dogs with mammary gland cancer (50 heads), normal dogs (50 heads), and benign tumor dogs (7 heads) were selected as subjects, and plasma was extracted therefrom. Thereafter, to remove albumin and IgG, which are abundant proteins in plasma (50 μl), solvents A and B of a multiple affinity removal kit (MARs) (Agilent) were used. At this time, albumin and IgG were removed while passing 800 μl of solvent A through a MARs column. In this way, when the amount of albumin present in plasma was reduced to 10% or less, the plasma samples were concentrated and dried using a vacuum concentrator (Speedvac). The concentrated and dried plasma samples were resuspended using 200 μl of HPLC-grade purified water, and then protein digestion was performed using a filter aided separation preparation (FASP) to remove unnecessary proteins. The protein digestion was carried out in the presence of trypsin for 16 hours, and after reaction, the proteins were frozen and stored. Thereafter, the stored samples were divided into three samples for analysis using a stop and go extraction (STAGE) tip containing a styrenedivinylbenzene-reverse phase sulfonate (SDB-RPS) filler, respectively. The separated final samples were analyzed using a mass spectrometer (Orbitrap fusion). After analysis, initial data were manually inspected to confirm that each sample was sufficiently found, and proteins contained in each sample were identified and quantified using a proteomic detection program (Maxquant) and a proteomic quantification program (perseus). To derive reliable results, the Maxquant analysis option was set to one or more unique peptides, six or more minimal amino acid lengths, oxidation (M) and Acetyl (Protein N-term) as expected PTM, and canine reference files were obtained from Uniprot.org.

### 1-2. Identification of Differentially Expressed Proteins

Among the proteins identified in Example 1-1, protein candidates, of which expression was increased or decreased, as compared with normal control groups, were found in blood samples of dogs with mammary gland cancer. The analysis was performed on proteins commonly identified in 90% or more of samples, including proteins commonly identified in all samples. Thereafter, a volcano-plot analysis using intensity of the expressed peptides was performed using proteins having a range of application of 90% to select differentially expressed proteins (p Value<0.05, fold change>1.2).

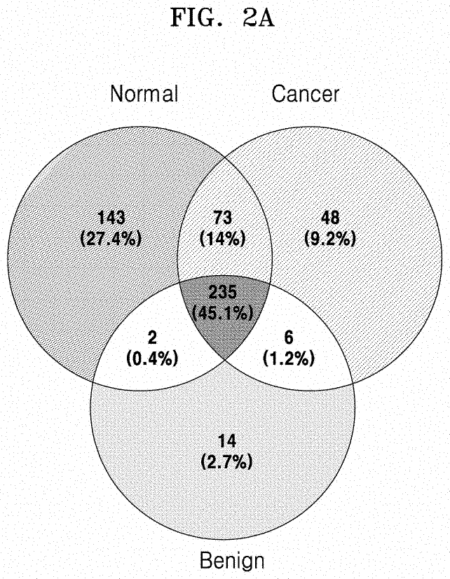

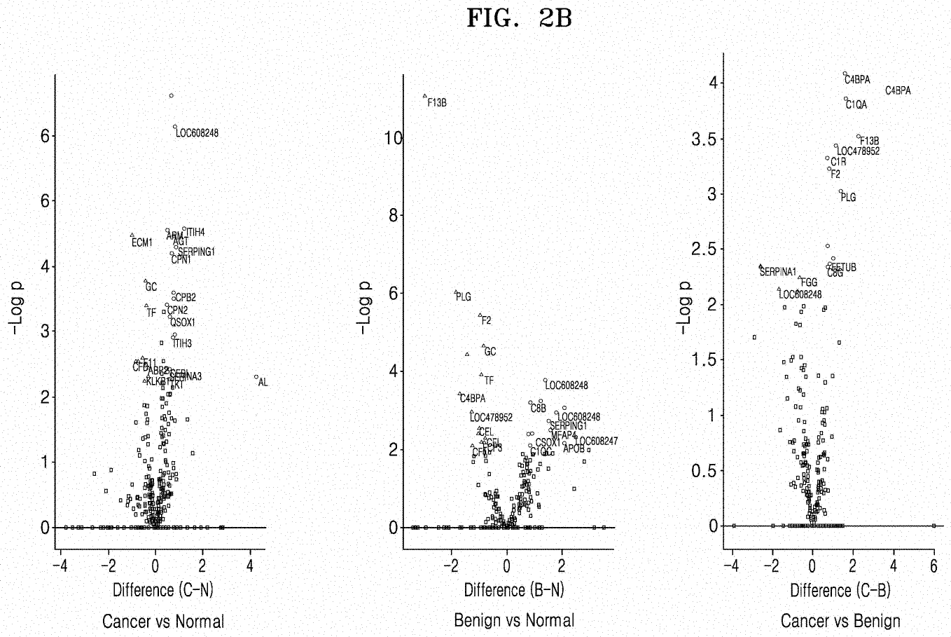

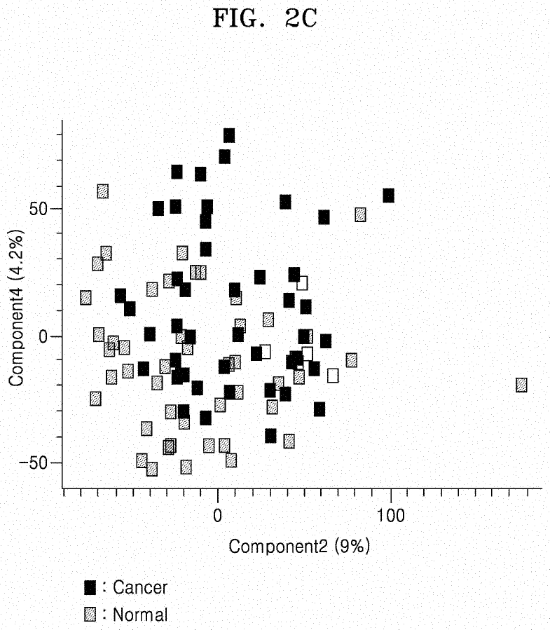

FIG. 2A shows a Venn diagram showing a relationship between proteins expressed in companion dogs with mammary gland cancer (cancer), normal dogs (normal), and dogs with benign tumors (benign).

FIGS. 2B and 2C show graphs showing proteins differentially expressed in companion dogs with mammary gland cancer (cancer), normal dogs (normal), and dogs with benign tumors (benign).

As a result, as shown in FIGS. 2B and 2C, it was confirmed that there were differences in the expressed proteins between companion dogs with mammary gland cancer and normal dogs, benign tumor dogs and normal dogs, and companion dogs with mammary gland cancer and benign tumor dogs.

### Example 2. Identification and Analysis of Human Breast Cancer-Related Protein Information, Based on Proteomics

### 2-1. Identification of Breast Cancer-Related Proteins

Blood proteins related to human breast cancer were identified in the same manner as in Example 1, except that breast cancer patients (31 patients) and normal control groups (19 patients) were targeted (see FIG. 1). A human reference file for proteomic analysis was obtained from uniprot-proteome_UP000005640.

### 2-2. Identification of Differentially Expressed Proteins

Among the proteins identified in Example 2-1, protein candidates, of which expression was increased or decreased, as compared with normal control groups, were found in blood samples of breast cancer patients in the same manner as in Example 1-2.

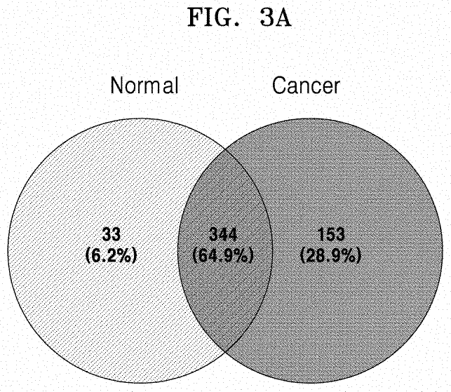

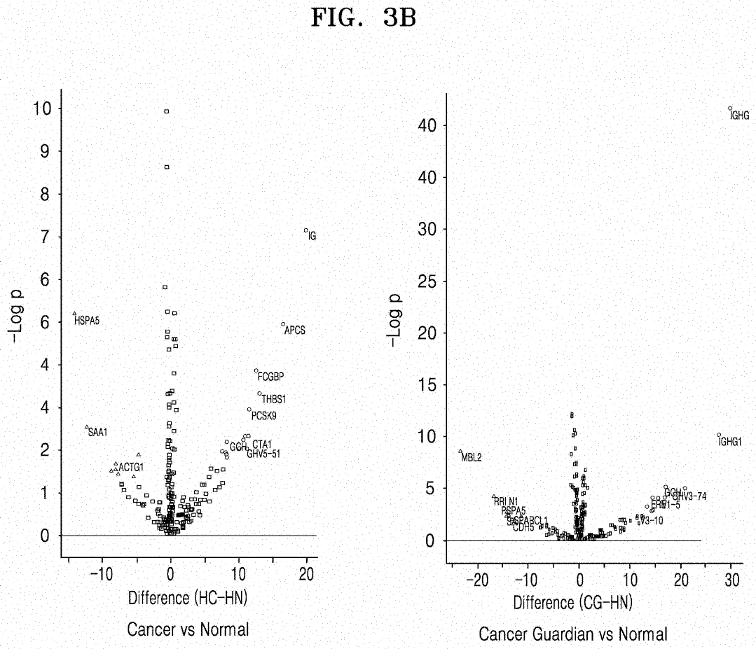

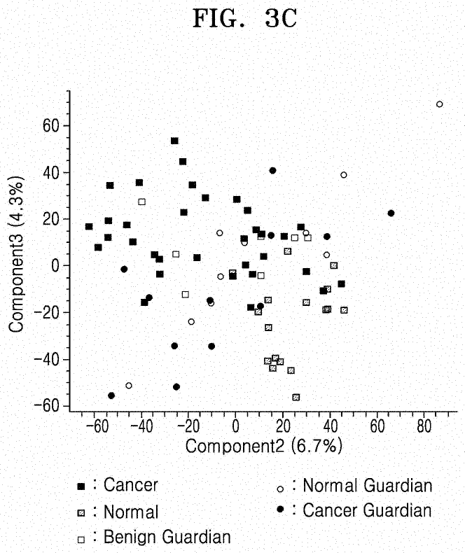

FIG. 3A shows a Venn diagram showing a relationship between proteins expressed in breast cancer patients (cancer) and normal control groups (normal).

FIGS. 3B and 3C show graphs showing proteins differentially expressed in breast cancer patients (cancer) and normal control groups (normal).

As a result, as shown in FIGS. 3B and 3C, it was confirmed that there were differences in the expressed proteins between breast cancer patients and normal control groups.

### Example 3. Identification of Changes in Protein Expression of Guardians Living with Companion Dogs with Mammary Gland Cancer

Based on the results of Examples 1 and 2, proteins of which expression levels were significantly increased or decreased in dogs with mammary gland cancer and breast cancers, as compared with normal control groups, were identified and shown in Table 2 below.

TABLE 2

| | Increase or decrease relative |
|---|---|
| Name of gene | to normal control group |
| SERPING1 | Increase |
| C1QA | Increase |
| LYZ | Increase |
| SEPP1 | Increase |
| FN1 | Increase |
| LCP1 | Increase |
| PROS1 | Increase |
| VWF | Increase |
| SHBG | Decrease |
| CFD | Decrease |
| PRDX2 | Decrease |
| HABP2 | Decrease |

The proteins shown in Table 2 are proteins that commonly show changes in their expression in dogs with mammary gland cancer and breast cancer patients when cancer develops. 12 kinds of the proteins were selected as biomarkers common among mammary gland cancer (dog) and breast cancer (human). Thereafter, the concept of companion dogs as sentinels was established by finding proteins commonly showing differences in their expression levels in the blood of guardians living with companion dogs with mammary gland cancer. The above concept applies that mammary gland cancer of a companion dog sharing the same living environment related to occurrence of breast cancer may be a diagnostic indicator for the guardian's breast cancer, because breast cancer is particularly affected by the environment, as compared with other cancers. First, serum samples were obtained from guardians (30 people) of companion dogs with mammary gland cancer, guardians (12 people) of companion dogs with benign tumors, and guardians (11 people) of normal companion dogs, and the blood proteins were analyzed in the same manner as in Example 1.

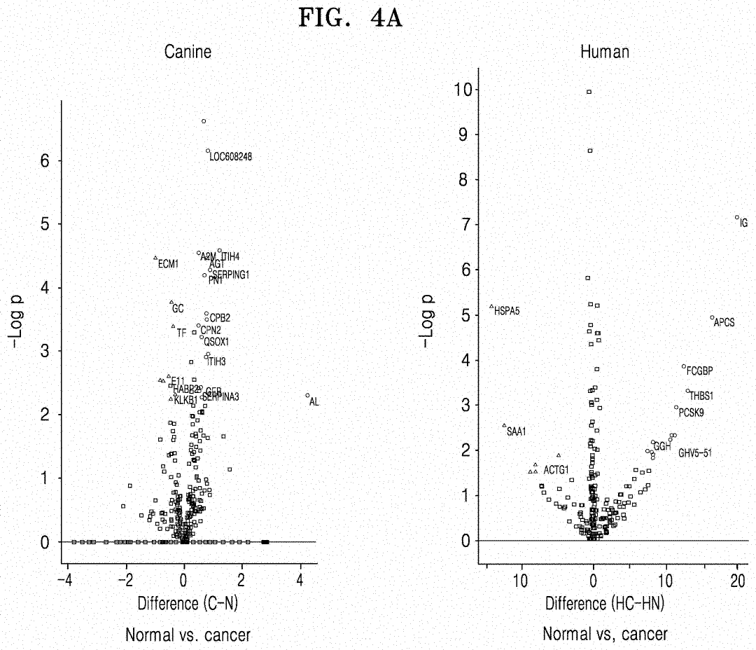

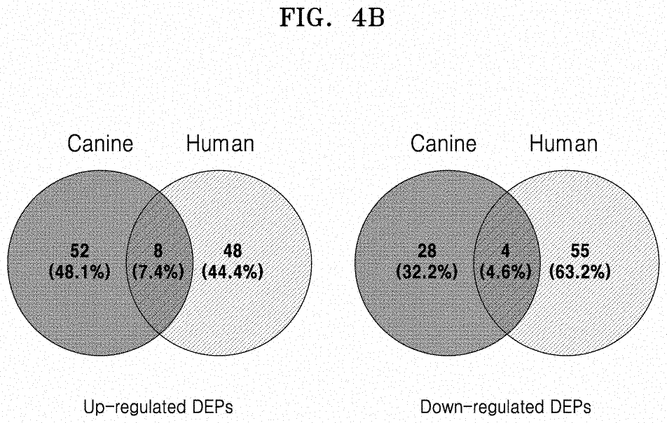

FIG. 4A shows graphs of proteins differentially expressed in companion dogs with mammary gland cancer, normal companion dogs, breast cancer patients, and normal control groups, respectively.

FIG. 4B shows a Venn diagram establishing proteins common among companion dogs with mammary gland cancer, normal companion dogs, breast cancer patients, and normal control groups, after identifying proteins differentially expressed therein by volcano plots, respectively.

As a result, as shown in FIG. 4A, it was confirmed that eight proteins were increased in the plasma of companion dogs with mammary gland cancer and patients with breast cancer, and four proteins were commonly decreased. Specifically, it was confirmed that expression of SERPING1, C1QA, LYZ, SEPP1, FN1, PROS1, LPC1, and VWF proteins was increased, whereas expression of SHBG, CFD, PRDX2, and HABP proteins was decreased.

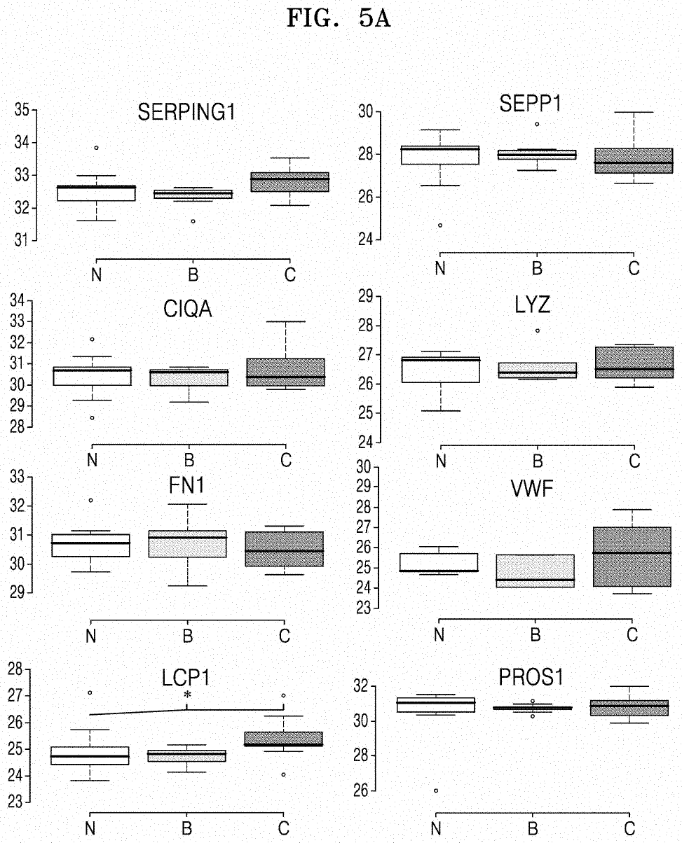

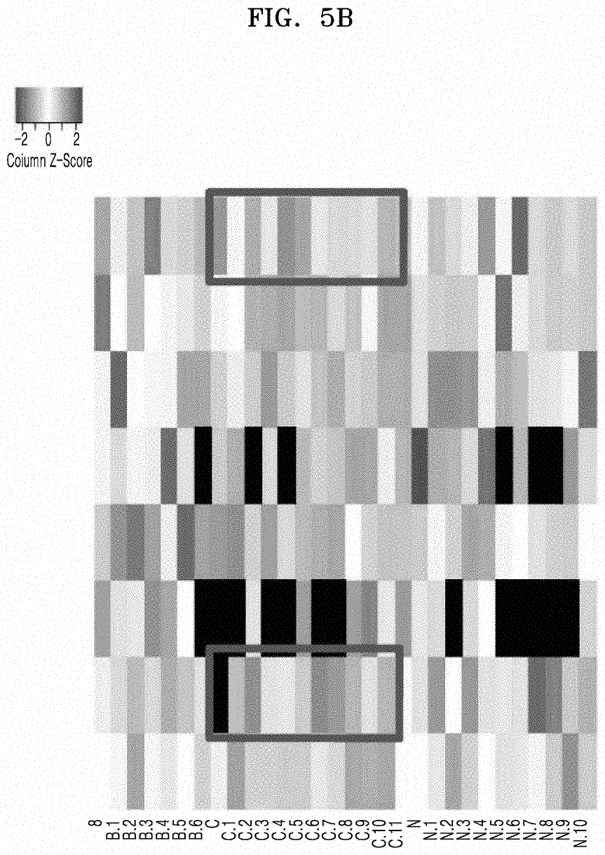

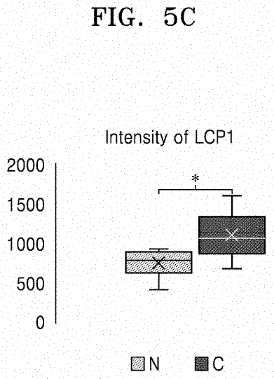

FIGS. 5A and 5B show expression patterns of eight kinds of proteins, of which expression was commonly increased in samples of guardians of companion dogs, companion dogs with mammary gland cancer, and breast cancer patients.

As a result, as shown in FIGS. 5A and 5B, among the eight kinds of proteins, LPC1 and SERPING1 were confirmed to be increased in the samples of guardians of companion dogs with mammary gland cancer. In particular, LPC1 protein was confirmed to be significantly increased in the samples of guardians of companion dogs with mammary gland cancer.

FIG. 5C shows a graph showing an expression pattern of LPC1, among the eight kinds of proteins, which was significantly increased in guardians of companion dogs with mammary gland cancer, as compared with guardians of normal companion dogs.

As a result, as shown in FIG. 5C, LPC1 expression was significantly increased in guardians of companion dogs with mammary gland cancer, as compared with guardians of normal companion dogs, and this LPC1 expression showed the same pattern as the plasma LPC1 protein increase in the breast cancer patients. In other words, LPC1 protein may predict not only the occurrence of mammary gland cancer in companion dogs, but also breast cancer of guardians of companions dog with mammary gland cancer, and thus it may be used as a sentinel marker.

According to a composition, kit, and method according to one aspect, a guardian's breast cancer may be diagnosed or predicted using a blood sample of a companion dog.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A composition for diagnosing breast cancer, the composition comprising an agent for measuring an expression level of LPC1 protein or mRNA of a gene thereof.

2. The composition of claim 1, further comprising an agent for measuring an expression level of SERPING1, C1QA, LYZ, SEPP1, FN1, PROS1, VWF, SHBG, CFD, PRDX2, or HABP2 protein, or mRNA of a gene thereof.

3. The composition of claim 1, wherein the agent for measuring the expression level of the protein is an antibody, an antibody mimetic, an aptamer, an avimer (avidity multimer), or a peptidomimetic specifically binding to the protein.

4. The composition of claim 1, wherein the agent for measuring the mRNA level of the gene is a primer pair, probe, or antisense oligonucleotide, which specifically binds to the gene.

5. A kit for diagnosing breast cancer, the kit comprising the composition of claim 1.

6. A method of providing information for diagnosing breast cancer, the method comprising:
measuring an expression level of LPC1 protein or mRNA of a gene thereof in a biological sample of an individual; and
comparing the measured expression level of the protein or mRNA of the gene with an expression level thereof in a normal control group.

7. The method of claim 6, further comprising measuring an expression level of SERPING1, C1QA, LYZ, SEPP1, FN1, PROS1, VWF, SHBG, CFD, PRDX2, or HABP2 protein, or mRNA of a gene thereof.

8. The method of claim 6, further comprising determining that breast cancer is present, when the measured expression level of the protein or mRNA of the gene is higher than that of a sample of a normal control group.

9. The method of claim 7, further comprising determining that breast cancer is present, when an expression level of SERPING1, C1QA, LYZ, SEPP1, FN1, PROS1, or VWF protein, or mRNA of a gene thereof is higher than that of a sample of a normal control group.

10. The method of claim 7, further comprising determining that breast cancer is present, when an expression level of SHBG, CFD, PRDX2 or HABP2 protein or mRNA of a gene thereof is lower than that of a sample of a normal control group.
